(19) United States
(12) Patent Application Publication
(10) Pub. No.: US 2022/0265912 A1
(43) Pub. Date: Aug. 25, 2022

(54) CONTINUOUS FLOW CATHETER ASSEMBLY AND METHODS OF USE

(71) Applicant: Sanford Altman, Miami, FL (US)

(72) Inventor: Sanford Altman, Miami, FL (US)

(21) Appl. No.: 17/183,274

(22) Filed: Feb. 23, 2021

(51) Int. Cl. A61M 1/28; A61M 1/16; A61M 1/26; A61M 39/10

(52) U.S. Cl. CPC A61M 1/285; A61M 1/1645; A61M 1/267; A61M 39/105

(57) ABSTRACT

A system includes a flexible catheter having at least one lumen, an adapter having an inlet, an outlet, at least one pump, and at least one channel in communication with the at least one lumen, the at least one pump being configured and arranged to move a fluid through the at least one lumen.

### FIELD OF THE DISCLOSURE

The present disclosure relates to catheters used in medical procedures. More particularly the present disclosure relates to a catheter and pump system designed for continuous-flow.

### BACKGROUND OF THE DISCLOSURE

It is estimated that the prevalence of chronic kidney disease in the United States population is 11% (roughly 19.2 million adult individuals) and increasing with more than 500,000 currently on maintenance dialysis. The kidneys are organs which function to extract water, urea, mineral salts, toxins, and other waste products from the blood. Patients with kidney failure require “dialysis,” a procedure that simulates the function of the kidneys in cleaning wastes and fluid from the blood, to stay alive.

There are currently two forms of dialysis available: hemodialysis and peritoneal dialysis. Hemodialysis is a well-known method of providing renal (kidney) replacement therapy by using a machine to remove excess fluid and waste products from the body. This is accomplished by removing blood, cleaning it and returning the cleansed blood back into the patient's body. During a hemodialysis treatment, blood is withdrawn from the patient's body, through a vascular access and then sent to the dialysis machine. In the dialysis machine, toxins and other waste products diffuse through a semi-permeable membrane into a dialysis fluid closely matching the chemical composition of the blood. The filtered blood (i.e., blood with the waste products removed) is then returned to the patient. As can be appreciated, proper access to the patient's vascular system for transport of blood to and from the dialysis machine is essential for hemodialysis to occur.

Currently there are three types of vascular access used for hemodialysis; catheters, grafts and fistulae. Grafts and fistulae are typically large diameter, fast flowing conduits located just beneath the skin surface. These conduits are typically punctured three times per week with two needles, something painful and not “ideal” as seen by patients. One needle removes blood from the patient's body and the second needle returns the cleansed blood back to the patient's body. These high flow conduits are known to have problems, most related to flow or lack thereof. Too high of flow can place excess work on the heart leading to significant cardiac issues for these patients, increasing their morbidity and mortality. High graft or fistula flow can also steal blood flow from the portion of the extremity distal to the access, typically the hand, causing vascular “steal” resulting in hand numbness, coldness, pain, weakness, non-functioning and possible amputation. To low of flow can result in graft or fistula clotting requiring clot removal procedures to restore these conduits into their needed flowing states. Whenever one of these Declot procedures is performed the patient is placed at increased risk of sending clot to areas of the body such as the arterial system thereby occluding arterial flow to a particular arterial distribution or to the pulmonary arterial bed causing cardiopulmonary disruption and/or collapse which can be fatal. Other issues such as stenoses (narrowings) and aneurysms (outpouchings) of the graft or fistula or their inflow arteries or outflow veins to name a few results in these types of access requiring frequent surgical procedures.

In more than 80% of the dialysis population, catheters are the first vascular access the patient will utilize. When used as the vascular access for dialysis the catheter consists of a tube, the catheter, which is placed through the skin, with its proximal (non-vascular) end with a connecting lock (lure lock) used to attached the catheter to the dialysis machine and to cover/close the catheter lumen with a cap when not in use and with its distal (intravascular) end positioned typically within a large vein within the body or the right atrium of the heart. When not in use for dialysis, unlike grafts or fistulae, catheters have no flow going through them. As such there are no high flow cardiac issues associated with catheters and there is no distal extremity steal. During a hemodialysis treatment, a catheter is connected directly to a dialysis machine and does not require the use of needles, preferable to most patients. The catheter may be a single tube with two or more separate lumens or two separate tubes. Generally, the length and diameter of the catheter will affect the catheter flow rates and pressures while performing dialysis. Maintaining functionality of an access is a major concern of dialysis because without vascular access the patient cannot dialyze.

Catheter dysfunction is often related to either clot or fibrin sheath. formation. Clot formation within the catheter lumen often occurs from stasis of blood, which forms clot, either from poor catheter flow, catheter tip and side hole designs or failure to property flush the catheter after use. Fibrin sheath formation often starts at the time of catheter placement, with tissue growth originating at the catheter entrance site and extending down and around the intravascular portion of the catheter, a foreign object residing within the intravascular space. Although why and how fibrin sheath eventually leads to occlusion of the catheter end holes where blood is aspirated and returned to and from the vascular system for dialysis is not clearly understood, typically it is something that does not occur during a normal dialysis treatment session itself when the catheter has flow running through it and is in use, removing blood, passing it through. the dialyzer and returning it to the patient. Obstructive flow limiting fibrin sheath typically occludes the end holes of the catheter lumen between treatments when the catheter is sitting idle, without flow.

Another problem seen with. catheters is infection. Catheter related bacteremia (CRBI)is a significant factor impacting morbidity and mortality in the dialysis population. Patients with catheters have significantly higher costs as well as poorer outcomes. Catheter infections typically occur in two ways, either through a break in sterile technique when placing the patient on or off of dialysis or from migration of an infecting organism from the catheter exit site to the subcutaneous space and/or vascular system. Infection from organism migration along the catheter tract is multifactorial related to; hygiene, catheter placement, tunneling techniques, catheter coatings and cuff designs. Currently, every time a catheter is used for dialysis the catheter caps must be cleaned and removed, opening the catheter and therefore the otherwise sterile vascular system to potential contaminates from air, hands and other surfaces prior to the catheter being attached to the sterile dialysis tubing/machine. At the completion of the treatment, the reverse process must occur, disconnecting the tubing from the catheter, exposing the vascular system to potential contaminates once again while flushing the catheter and reapplying the catheter caps. Each time this occurs, with each treatment, the patient is placed at risk, whether done by trained professionals at dialysis centers, or at home by caregivers or the patient themselves. This risk is not theoretical, but actual given the high risk of septic events that occur in End-Stage Renal Disease patients receiving hemodialysis through catheters.

Despite advances that have been made in providing vascular access for dialysis, there are a variety of problems associated with currently available catheters. For example, a significant problem with dialysis catheters is the risk of infection and clotting. The suction produced at the opening of a hemodialysis catheter can be occluded by intimal tissues (i.e., a fibrin sheath) within the blood vessel and result in clotting. Hence, there is a continuing need for an improved catheter that decreases the potential for fibrin sheath formation and reduces the risk of infection while allowing for more effective dialysis.

### SUMMARY OF THE DISCLOSURE

In some examples, a system includes a flexible catheter having at least one lumen, and an adapter having an outlet, at least one pump, at least one channel in communication with the at least one lumen, and the at least one pump being configured and arranged to move a fluid through the at least one lumen.

In some examples, a continuous flow adapter includes a housing defining an inlet, an outlet, and two channels, each of the two channels being configured and arranged to couple to one of two lumens of a catheter, and at least one pump disposed adjacent one or more of the two channels and configured and arranged to support blood flow through the one or more of the two channels.

### BRIEF DESCRIPTION OF THE DISCLOSURE

Various embodiments of the presently disclosed catheters are disclosed herein with reference to the drawings, wherein:

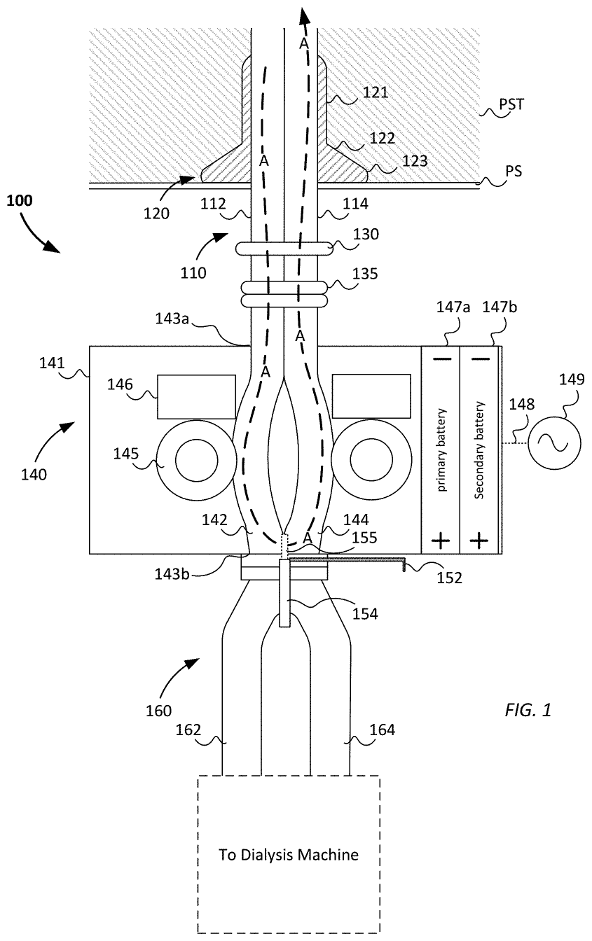

FIG. 1 is a schematic cross-sectional illustration of a system including a catheter, an adapter and connection to a dialysis machine;

FIG. 1 is a schematic cross-sectional illustration of a system including a catheter, an adapter and connection to a dialysis machine, and a first blood flow circuit;

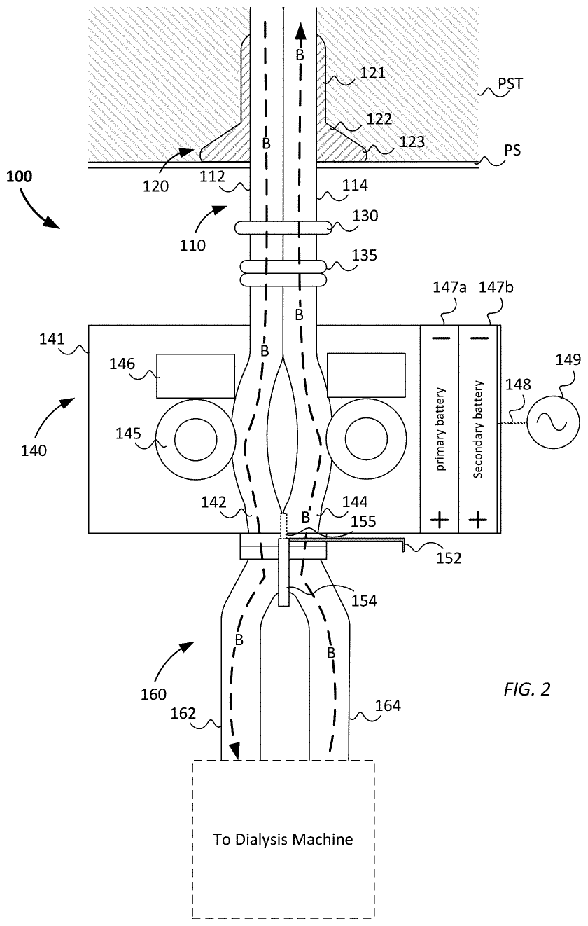

FIG. 2 illustrates a second blood flow circuit through the system of FIG. 1;

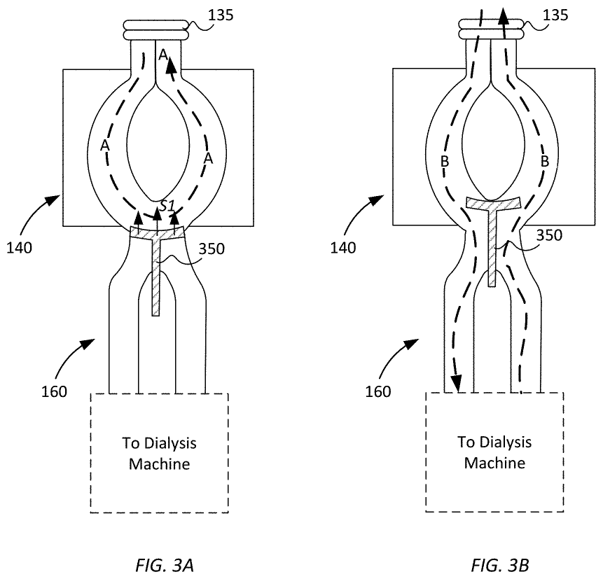

FIGS. 3A-B illustrate a cross-section view of an adapter having a splitter in a closed an open condition, respectively;

FIG. 3C illustrates another example of an adapter having a single pump; and

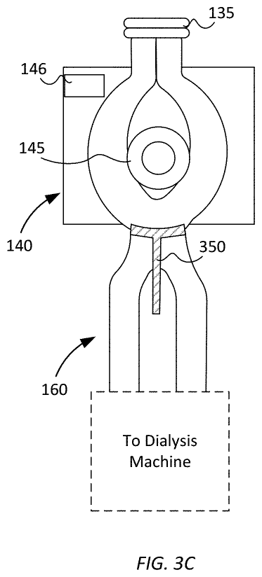

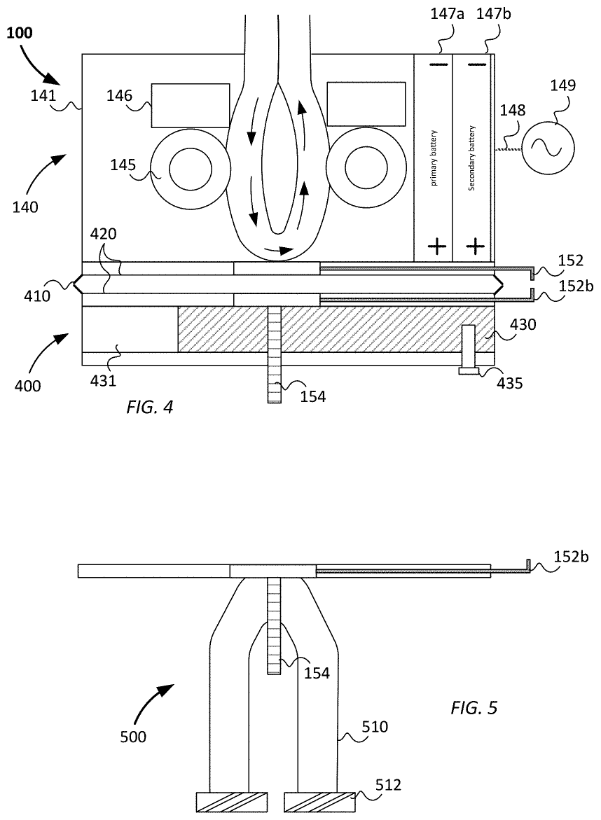

FIGS. 4-5 illustrate schematic illustrations of a safety cap and a clot checker cap.

Various embodiments of the present invention will now be described with reference to the appended drawings. It is to be appreciated that these drawings depict only some embodiments which may or may not all be required for functionality of the invention and are therefore not to be considered limiting its scope.

### DETAILED DESCRIPTION

Despite the various improvements that have been made to catheters, conventional devices suffer from some shortcomings as described. above.

There therefore is a need for further improvements to the devices, systems, and methods of vascular access for hemodialysis. Among other advantages, the present disclosure may address one or more of these critical needs.

As used herein, the term “proximal,” when used in connection with a component of a catheter assembly, refers to the end of the component closest to the physician, the patient and others when the catheter is inserted in a patient, whereas the term “distal,” when used in connection with a component of a catheter assembly, refers to the end of the component farthest from the physician residing or intended to reside within the desired location within vascular system. Likewise, the terms “trailing” and “leading” are to be taken as relative to the operator (e.g., physician) of the catheter assembly. “Trailing” is to be understood as relatively close to the operator, and “leading” is to be understood as relatively farther away from the operator.

In some embodiments, continuous flow catheter system provides continuous uninterrupted flow within. the system and access to the blood system for prolonged and/or chronic therapies when needed. Generally, the system may include a catheter and a pump system. Although described here with regards to End-Stage Renal Disease and hemodialysis, it will be understood that the devices, methods and techniques of this disclosure are not so limited, and that the teachings may be used in other treatments that would benefit from continuous fluid movement within a catheter or organ system, vascular or non-vascular.

Despite advances in dialysis and vascular access, placing and maintaining vascular access still plagues the patient population. Most recent USRDS data shows that the average patient receiving hemodialysis requires close to two vascular access procedures per year to keep their access functioning properly without complications. Vascular access places a significant healthcare risk and financial burden on healthcare systems and patients. The uniqueness of the continuous flow catheter is in its ability to maintain continuous flow, designed specifically to address some of the known problems with current vascular accesses. Different from graft and fistula, the continuous flow catheter will not have high flow and thus will not have any of the associated complications associated with high flow graft and fistulae. When used as intended it will not have large clot volume and will not have any of the complications seen when declotting graft and fistulae. Aneurysms and stenoses, other issues requiring frequent surgical intervention with graft and fistula will not be an issue with this device. In comparison with current catheter technology all of which remain dormant when not connected to the dialysis machine, the continuous flow catheter will reduce if not prevent catheter end hole or side hole obstructive fibrin sheath formation, the most common cause of catheter dysfunction and failure. Continuous flow within the catheter may also reduce the risk of infectious (ie. bacterial) colony growth, something that can occur when the catheter accidentally receives an infectious agent at its end of treatment and then remains dormant for days until its next use giving the infectious agent time for multiplication, growth and colonization within the catheter lumen all the way to the catheter tip where the infectious agent can become incorporated into the biofilm/fibrin sheath. Additionally, Cuff design and dialysis machine connections, as perfected by one skilled in the art, will be created to lower the infection risk of this catheter as compared with non-continuous flow catheters. Additionally, the continuous flow within the catheter may reduce the risk of bacterial colonization at ends of the catheter.

FIG. 1 illustrates a continuous catheter system 100 that generally includes a catheter 110, a cuff 120, connection means 135, an adapter 140 and a disposable tubing 160 for coupling to a dialysis machine. Catheter 110 may include two single lumen catheters, or a dual-lumen catheter in a side-by-side, circle C, Double D or Coaxial configuration to name a few that includes a first lumen 112 adjacent a second lumen. 114. It will be understood that the configurations of the catheter may be modified by one skilled in the art. Catheter 110 is intended to be at least partially disposed within the patient's body, a portion of the catheter extending out through. patient subcutaneous tissue “PST” and the patient's skin “PS”. A cuff 120 is disposed about the catheter and configured to anchor the catheter (e.g., cuffed tunneled catheters) within the patient's body and prevent accidental dislodgement of the catheter that would displace or remove it from its intended location within the body. Cuff 120 may be useful for allowing subcutaneous tissue to grow into it and to help secure the catheter once it is implanted in a patient's body. Cuff 120 may be composed of polyester, Dacron® polyester or other suitable materials. In one possible cuff design, the embodiment shown, cuff 120 includes a generally cylindrical segment 121 that flares out to a transition 122 where its outer diameter increases until it reaches a larger diameter segment 123 closer to the patient's skin. This cuff configuration 120 may provide a larger surface area for tissue ingrowth, and the large diameter segment 123 may better anchor the catheter within the patient's body. In this manner, a cuff 120 having a non-constant outer diameter may be used to anchor the catheter at the appropriate position in a more secure manner than cuffs currently in use. Additionally, this cuff 120 may reduce the risk of tunnel tract infections through increasing surface area for tissue ingrowth. In some examples, the catheter cuff may be further anchored via suturing to a suture wing built into the cuff.

Turning back to catheter 110, a safety clamp 130 is disposed about, or otherwise coupled to, catheter 110 at its proximal end (i.e., closer to the physician). Safety clamp 130 may be used in case of emergency to shutoff flow through the catheter. Adjacent safety clamp 130, a catheter-pump connector 135 may be disposed, which allows for the coupling of the catheter 110 to channels of adapter 140. In some examples, connector 135 is not removable unless required for either pump replacement/removal of catheter repair, replacement, or removal. Connector 135 may be constructed in a manner to prevent leakage and maintain sterility, and may, for example, include a luer-lock assembly

Adapter 140 may include a housing 141 that encloses a pair of channels 142,144, the first channel 142 being in. fluid. communication with first lumen 112, and the second channel 144 being in fluid communication with second lumen 114 via connector 135. As shown, channels 142,144 are partially disposed within housing 141 of adapter 140, and partially outside the housing extending through adapter inlet 143a, although. other configurations are possible. One or more roller or peristaltic pumps 145 may be disposed adjacent each of the channels 142,144, and configured to impinge on the flexible channels to drive fluid therethrough. Two pumps are shown in the embodiment of FIG. 1, although it will be understood that a single pump may be used as shown in FIG. 3C or multiple pumps if needed as determined by one skilled. in the art. Pumps 145 may be driven by one or more motors 146. Other kinds of pumps are possible as best designed by those skilled in the art. In at least some examples, the pumps 145 are configured to maintain blood flow through the channels at a desired fluid flow rate which may be fixed or adjustable to best serve patient needs. As used herein, the term “continuous” means that blood flow through the lumen occurs even when the catheter is not connected to a dialysis machine. In at least some example, the pump works in an uninterrupted fashion. Alternatively, the pumps of the adapter may operate semi-continuously at regular intervals. For example, and for some healthcare needs the pump may be driven for a period of time (e.g., for 5 minutes) with a predetermined period (e.g., every hour). Adapter 140 may pull blood, or other fluid, from one lumen of the catheter while returning blood, or other fluid through the other with the ability to reverse flow direction if desired. In some examples, adapter 140 may function in either AC or DC modes. Additionally, adapter 140 may be as small and light weight as possible to provide optimal flow at minimal demands to allow for maximum battery life for patient use.

The adapter 140 maybe powered by a primary battery 147a and may include a secondary battery 147b. These batteries may be rechargeable or replaceable, and a wiring 140 may be used to recharge the batteries from a power source 149. In some examples, the dual battery system may be used to provide a safety mode for additional power when the primary battery 147a discharges. The primary battery may be exchangeable to allow for recharging and replacement of the uncharged battery with the back-up or secondary battery 147b providing uninterrupted flow while a new primary battery 147a is installed. Batteries may be rechargeable while in use or when removed when connected to the AC current with the AC current providing power to both the pump(s) and battery charger.

Adapter 140 may be coupleable to a disposable tubing 160 that includes a first branch 162, and a second branch 164, the disposable tubing 160 being coupleable to a dialysis machine or other medical device. Although the adapter 140 is being illustrated in use with a dialysis machine, it will be understood that adapter 140 may be coupleable to a tubing or other connection of another medical device. In at least some examples, a fluid-flow routing mechanism between the adapter 140 and the disposable tubing 160 is provided to permit or occlude flow to a dialysis machine. In one example, the fluid-flow routing mechanism. includes a first lever 152, and a second lever 154 that provide an openable and closeable gate 155 to divert fluid between one of two circuits. First lever 152 may be the form of a sliding door used to open a chamber of the adapter 140 to allowing blood to flow through the disposable tubing 160. Second lever 154 may be used to divert flow from the catheter and adapter 140 to the disposable tubing 160 and the dialysis machine. In at least some examples, first lever 152 may be actuated. only when a disposable tubing 160 is attached to the adapter 140. In at least some examples, second lever 154 may be actuated or depressed only after the first lever 152 is opened or actuated so that sequential operation is needed.

With the adapter 140 attached. to catheter 110 via catheter-pump connector 135, two possible circuits exist based on the position of first and second levers 152,154. First, without a disposable tubing or other attachment to a dialysis machine, blood may flow through first lumen 112 of catheter 110, first channel 142 of adapter 140, through open. gate 155, back through second channel 144 of adapter 140 and second lumen 114 of catheter 110. This is “short circuit” illustrated and labeled as circuit “A” in FIG. 1.

FIG. 2 illustrates the second possible circuit, where blood. flows through the pump to the dialysis machine. With the adapter 140 attached to catheter 110 via catheter-pump connector 135, the disposable tubing 160 coupled to the adapter 140, and the levers 152,154 properly actuated, the second, long circuit “B” is possible. First, blood may flow through first lumen 112 of catheter 110, first channel 142 of adapter 140 then leave adapter 140 through an outlet 143b of the adapter into a first branch 162 of disposable tubing 160 to be processed through the dialysis machine. Blood. may then return via second branch 164 of disposable tubing 160, then back through second channel 144 of adapter 140 and second lumen 114 of catheter 110. Of note, while the adapter has been described as having an inlet 143a, and an outlet 143b, it will be appreciated that blood may flow through in and/or out of these ports in either direction depending on the chosen circuit and direction of blood flow. When flowing through the dialysis machine the continuous flow catheter pump may or may not be used for blood flow.

The system 100 may be used to remove, clean and return the blood in center hemodialysis, home hemodialysis, nocturnal hemodialysis, daily hemodialysis or continuous hemodialysis. System 100 brings patients closer to the ideal access, one that is painless to use, has no needle stick complications, has no cardiac related or distal arterial seal related high flow concerns, has a reduced risk of catheter dysfunction and failure and a reduced risk of catheter-related blood stream infections. Whether used for access during traditional in-center dialysis or at home by caregivers or patients themselves for home hemodialysis or for continuous ambulatory dialysis the system is designed to reduce the risks associated with vascular access.

Specifically, system. 100 may provide continuous, uninterrupted flow to maintain superior patency as compared with current access options available for hemodialysis. System 100 may maintain constant flow and patency through the use of adapter 140 when the catheter is not in its therapeutic mode, and/or from pumps of a dialysis machine when the system is being used for therapy. The system 100 may reduce and/or prevent fibrin sheath formation and concomitant flow disruption, different from all other catheters used for dialysis due to its unique constant. flow. An additional benefit from constant flow, as compared. to constant flow seen in grafts or fistulae, is that the blood flow through this system will be significantly less than the Kidney Disease Outcomes Quality Initiative (KDOQI) minimum recommendations of 600 cc/min for grafts and fistula, reducing the cardiovascular workload, morbidity and mortality seen in high flow grafts and fistulae. The system tip may reside in its preferred location as positioned by the implanter, which for hemodialysis is typically the right atrium or caval-atrial region, allowing for optimal flow. The extravascular portion of the catheter may be tunneled in a manner to allow the proximal end of the catheter to exit the subcutaneous space near the desired positioning of the pump adaptor 140, as determined by the implanter and those skilled in the art.

Variations, for example, of the fluid flow routing mechanism are possible. In some embodiments, the system may include a double lever or door design on adapter 140 to allow or impede blood flow between adapter 140 and disposable tubing 160. A single door configuration is possible, but two doors may be added for safety, both of which may be activated by the connector allowing the doors to be moved into the open position once the connector is in place. In some examples, the connector may contain a moveable T-shaped or Y-shaped splitter 350, which when moved in the direction of arrows “S1” may be used to divert the normal flow within the adapter to flow to the disposable tubing to create two possible circuits, A and B, one to bring blood from the patient through the catheter and adaptor through the disposable tubing to the dialysis machine and the other (B) to return the blood from the dialysis machine through the tubing, adaptor and catheter back to the patient. (FIGS. 3A-B).

In some embodiments, the system 100 will also include a safety cap 400 capable of functioning as a bacterial barrier and/or serving as a safety flush system in case of battery failure. Turning to FIG. 4, safety cap 400 may couple to adapter 140 at its lever end via locking pins 410 to cover and secure the lever end after each dialysis treatment. In some examples, safety cap 400 may click onto the lever end of the adapter 140 using pins, hinges, or other suitable coupling members. In its initial placement, safety cap 400 may lock both levers in place keeping the first lever 152 and associated cap lever 152b to the blood chamber locked, and the flow diverting second lever 154 (lever 2) in its extended, non-flow-diverting position, allowing for continuous flow between the catheter 110 and adapter 140. In some embodiments, presenting surface(s) 420 between safety cap 400 and adapter 140 may be coated with bacteriostatic material to maintain a sterile environment in this capped space. In yet another embodiment an electronic bacteriostatic means may be utilized to maintain sterility within this interface between safety cap and pump.

In some embodiments, a safety chamber 430 may be included in safety cap 400, the chamber 430 being prefilled with fluid (e.g., saline). The purpose of this chamber, with a corresponding filling mechanism 435, either manual or electronic, will be to fill and replace the blood-filled channels 142, 144 of adapter 140 and catheter lumens 112, 114 with saline, pushing out the blood prior to pump/battery failure preventing clot formation within the continuous flow catheter system. In some example, this feature may only be able to be utilized prior to battery or power discharge as controlled by the system electronics. Once the system powers down, a mechanism may be included to prevent the system from powering back up until the pump has been evaluated and verified by a healthcare professional to ensure that no clot is present in the catheter lumen 112, 114 or adaptor channel 142,144.

In another embodiment, cap 400 may have a secondary medication chamber 431 capable of being filled or prefilled with medication or therapeutics capable of being released into the channels 142,143 at a preset manner and rate as controlled by the system/adaptor processor and/or as determined by the patient and/or healthcare provider. In some examples, safety chamber 430 and medication chamber 431 may be separate compartments and may retain different fluids. This mechanism could allow for timed release of medications/therapeutics through such continuous flow catheter.

Prior to system power failure, at a selected time prior to battery discharge, the system may present an alarm or notification indicating failure within a given time. In one configuration the alarm can be reset for a desired interval to stop the alarm while battery/power issues are being examined. If AC or DC power options are not available safety chamber 430 may be activated, allowing the operator to stop the pump, open the first and second levers 152,154 from mechanisms within the safety cap 400 and then fill the system with saline displacing any/all blood products from the system back into the patient. In cases of system power failure, the saline will prevent the system from clotting.

Once the safety chamber 430 has been activated the adaptor 140 pump will be prevented from being re-activated until a new disposable connector “clot checker” cap 500 is applied. The pre-filled therapeutic chamber, if present, will also not function during this time. This cap 500 will allow the operator to aspirate from, or flush, both catheter lumen 112, 114 as well as the adaptor channels 142,144 to ensure no clot is present within the continuous flow catheter system (FIG. 5). In some examples, the clot checker/flush cap 500 may include the same connecting cap design to connect to adapter 140 with similar lever controls 152b and 154 and will be applied to the pump once the safety cap 400 has been removed. Clot checker/flush cap 500 may include tubings 510 with lure lock endings 512 to allow fluid and/or blood to be aspirated and returned through both lumen of the adaptor chambers 142, 144 and the catheter lumen 112, 114 to ensure no clot is present. Once the absence of clot is confirmed, either a new safety cap 400 may be applied and the pump restarted or a new dialysis disposable tubing 160 can be attached to begin a dialysis treatment.

In some embodiments, catheter 110 and adapter 140 are unitarily formed. Alternatively, a retrofitting adapter having a pump may be coupleable to a conventional catheter to provide the continuous flow capabilities. While the adapter 140 has been illustrated herein as being disposed outside of the body, it will be understood that variations are possible in which the pump is implanted in the body or underneath the patient's skin. In some examples, the adapter 240 is implanted at the same site, or adjacent to the cuff 120. Alternatively, the adapter and cuff 120 may be implanted at separate sites.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

It will be appreciated that the various dependent claims and the features set forth therein can be combined in different ways than presented in the intial claims. It will also be appreciated. that the features described in connection with individual embodiments may be shared with others of the described embodiments.

What is claimed is:

1. A system comprising:
a flexible catheter having at least one lumen; and
an adapter having an inlet, an outlet, at least one pump, and at least one channel in communication with the at least one lumen, the at least one pump being configured and arranged to move a fluid through the at least one lumen.

2. The system of claim 1, wherein the at least one lumen includes two lumens, and the at least one channel includes two channels, each of the channels being in communication with one of the two lumens.

3. The system of claim 2, wherein the at least one pump includes two roller pumps configured to move fluid through the two channels.

4. The system of claim 1, further comprising at least one motor configured to drive the at least one pump.

5. The system of claim 4, wherein the adapter further comprises a fluid-flow routing mechanism configured to divert flow through one of two circuits.

6. The system of claim 5, wherein the two circuits include a first circuit defined by a route through a first of the two lumen, a first of the two channels, and returning through a second of the two channels, and a second of the two lumens.

7. The system of claim 5, wherein the two circuits include a second circuit defined by a route through a first of the two lumen, a first of the two channels, the outlet, a first branch of a tubing, a dialysis machine, and returning through a second branch of the tubing, a second of the two channels, and a second of the two lumens.

8. The system of claim 5, wherein the fluid-flow routing mechanism comprises at least one lever being capable of opening and closing a gate between the first and second channel.

9. The system of claim 5, wherein the fluid-flow routing mechanism comprises a splitter of various shapes including Y-shaped or T-shaped splitter.

10. The system of claim 1, further comprising a safety clamp to shutoff flow through the flexible catheter, and a catheter-pump connector for coupling the at least one channel to the at least one lumen.

11. The system of claim 1, wherein the adapter further comprises at least one battery configured to power the at least one pump.

12. The system of claim 1, further comprising a safety cap coupleable to the adapter adjacent the outlet.

13. The system of claim 12, wherein the safety cap includes a fluid-filled chamber and a medication chamber.

14. The system of claim 1, further comprising a clot checker coupleable to the adapter adjacent the outlet.

15. The system of claim 1, wherein the flexible catheter further comprises a cuff for promoting subcutaneous tissue ingrowth, the cuff including a generally cylindrical segment having a first diameter, a transition segment and an enlarged segment having a second diameter larger than the first diameter.

16. A continuous flow adapter, comprising:
a housing defining an inlet, an outlet, and two channels, each of the two channels being configured and arranged to couple to one of two lumens of a catheter; and
at least one pump disposed adjacent one or more of the two channels and configured and arranged to support blood flow through the one or more of the two channels.

17. The continuous flow adapter of claim 16, wherein the at least one pump includes two roller pumps.

18. The continuous flow adapter of claim 16, wherein the adapter further comprises a fluid-flow routing mechanism configured to divert flow through one of two circuits.

19. The continuous flow adapter of claim 18, wherein the two circuits include a first circuit defined by a route through a first of the two lumens of the catheter, a first of the two channels, and returning through a second of the two channels, and a second of the two lumens of the catheter.

20. The system of claim 18, wherein the two circuits include a second circuit defined by a route through a first of the two lumens of the catheter, a first of the two channels, the outlet, a first branch of a tubing, a dialysis machine, and returning through a second branch of the tubing, a second of the two channels, and a second of the two lumens of the catheter.
